(19) United States
(12) Patent Application Publication
(10) Pub. No.: US 2004/0248769 A1
(43) Pub. Date: Dec. 9, 2004

(54) Enzyme inhibitors for inactivating allergens

(76) Inventors: Claes Bavik, Seattle, WA (US); Michael Cork, Sheffield (GB); Birgit Helm, Sheffield (GB); David Buttle, Sheffield (GB)

Correspondence Address: Thomas M Galgano, Galgano & Burke, 300 Rabro Drive, Suite 135, Hauppauge, NY, 11788, US

(21) Appl. No.: 10/487,657

(22) PCT Filed: Oct. 24, 2002
(86) PCT No.: PCT/GB02/04841

(30) Foreign Application Priority Data
Oct. 25, 2001 (GB) 0125594.2

(51) Int. Cl. A61K 38/56

(52) U.S. Cl. 514/2

(57) ABSTRACT

The inventive concept relates to the inactivation of allergens using inhibitors for hydrolytic enzymes, and preferably proteases. Embraced within the overall inventive concept is the use of hydrolytic enzyme inhibitors for manufacture of medicaments for the prophylactic treatment of allergic conditions, and formulations for their use. Also, there is included a method for the manufacture of air treatment devices with the enzyme inhibitor bound to the surface of the support material such as an air filter. There is also a formulation containing the enzyme inhibitors for inactivating allergenic residues on fabrics or carpets.

### FIELD OF THE INVENTION

The invention relates to the inactivation of allergens preferably (but not exclusively) using proteinase (protease) inhibitors.

### REVIEW OF THE ART KNOWN TO THE APPLICANT(S)

Previous efforts to minimise allergic responses have relied on modifying the immune response of an individual following exposure to an allergen. Enormous efforts have been devoted to pharmaceutical therapies which interfere with the allergic response, once it has been initiated. However relatively little effort has been devoted to preventing the initiation of an allergic response.

Clearly avoiding the allergy inducing allergen is one way of preventing the initiation of an allergic immune response/reaction, but even if the identity of the allergy inducing allergen is known, avoidance is not always possible (for example in the case of dust mite induced allergies) or desirable (for example for animal lovers who experience allergies to animal borne allergens).

Little effort has, to the applicant's knowledge been devoted to seeking to render known allergens immunoloically inactive and/or to reduce their allergenic/immunogenic properties.

It has been established that many allergens are hydrolytic enzymes such as phospholipases, proteinases or lectins which are capable of activating cells of the innate immune system by cleaving or crosslinking cell surface receptors and stimulating the synthesis of pro-inflammatory cytokines before adaptive immune responses occur.

Evidence that the allergenicity of these enzymes is related to their enzymatic activity is now very strong.

The invention resides in the realisation that inhibiting the enzymic activity of these substances will prevent the establishment of an allergic response in susceptible individuals.

More specifically the appreciation by the inventors that known proteolytic allergens can be effectively inactivated using proteinase inhibitors to prevent the initiation of an allergic immune response constitutes a significant part of the invention now claimed.

Examples of some suitable/preferred synthetic proteinase inhibitors for use in the invention include peptide aldehydes, epoxidyl peptides, diazomethanes, chloro- and fluoromethanes, vinyl sulfones, acyloxymethylketones, isocoumarins, and phosphonates etc.

Summary of the Inventive Concept and Linked Inventions

In its broadest aspect, the inventive concept provides the use of inhibitors to inhibit the immunological activity of allergens. By way of example we have assayed the proteolytic activity of potential allergens extracted from the pelt or present in the saliva of a number of pets or domestic animals, and demonstrated inhibition by certain proteinase inhibitors.

It will be immediately apparent to the skilled addressee that the same approach applies equally to the inactivation of other proteolytic allergens for example those produced by house dust mites, cockroaches, washing powders, detergents, or pollen.

In its broadest aspect, the first invention provides the use of a hydrolytic enzyme inhibitor for the manufacture of a medicament for the prophylactic treatment of allergic conditions. Preferably, the hydrolytic enzyme inhibitor is a proteinase inhibitor.

In either of the above uses, the allergic condition preferably causes one or more of the following symptoms: shortness of breath, hyperventilation, sneezing, inflammation of the mucus membranes, skin rashes, nasal congestion.

Another aspect of the invention is a formulation for use in any of the preceding inventions comprising a proteinase inhibitor (wherein the proteinase inhibitor is non-toxic and non-allergenic).

Preferably also, a dry powder formulation for use in any of the above uses or formulations, comprising a proteinase inhibitor which optionally becomes active on wetting.

Preferably also, a liquid formulation for use in any of the above uses or formulations, comprising a proteinase inhibitor and optionally a cleansing agent.

More preferably, the use or the formulations described above, wherein the hydrolytic enzyme inhibitor inhibits arginyl endopeptidase activity.

More preferably also, the use or the formulations described above, wherein the hydrolytic enzyme inhibitor serine proteinase activity.

In its broadest aspect, the second linked invention forming the inventive concept provides a method of manufacture of an air treatment device for inactivating airborne allergens wherein a hydrolytic enzyme inhibitor is bound to a supporting medium, said supporting medium being brought into contact with the air during use.

Preferably, in the method of the second linked invention, the hydrolytic enzyme inhibitor is a proteinase inhibitor.

More preferably, in the method of the second linked invention, the hydrolytic enzyme inhibitor inhibits arginyl endopeptidase activity.

More preferably also, in the method of the second linked invention, the hydrolytic enzyme inhibitor inhibits serine proteinase activity.

In any of the methods of the second linked invention, the said supporting medium is maintained in a moist environment during use.

In its broadest aspect, the third linked invention forming the inventive concept provides a formulation for inactivating allergenic residues on fabrics or carpets comprising a hydrolytic enzyme inhibitor.

Preferably, the hydrolytic enzyme inhibitor of the formulation of the third linked invention comprises a proteinase inhibitor.

Preferably also, the hydrolytic enzyme inhibitor of the formulation of the third linked invention inhibits serine proteinase activity.

Preferably also, the hydrolytic enzyme inhibitor of the formulation of the third linked invention inhibits arginyl endopeptidase activity.

### DESCRIPTION OF THE PREFERRED EMBODIMENT

The invention will be described by way of example with reference to the following tables in which:

Table 1 is a descriptive list of the samples tested.

Table 2 is a table showing the results of analysis of the proteolytic activity associated with the extracted fur and saliva samples.

Table 3 is a table showing the rates of cleavage of Z-Gly-Gly-Arg-NHMec and Z-Phe-Arg-NHMec by the extracted cat fur samples.

Table 4 is a table showing the effect of a variety of protease inhibitors on the proteolytic activity of one of the extracted cat fur samples.

Table 5 shows the determination of Kᵢ for the inhibition of Catl S3 by leupeptin.

At least 10% of the Western population experience some form of allergic immune reaction to cats. The allergen is thought to be derived from the saliva of the cat and is transferred to the pelt during the cat's regular cleaning chores. Allergen is also thought to be secreted by the lacrymal, skin and anal sebaceous glands.

When in solution the allergen is unlikely to evoke an allergic response even in highly susceptible individuals. After a cleaning session however allergen dries on the pelt, this dried allergen is released and easily becomes airborne as the animal moves, scratches or is petted. Airborne allergen is much more likely to cause an allergic immune response in susceptible individuals, than the dissolved form.

Although the cat allergen Fel d1 was sequenced in 1991 by Morgenstern JP, et al and no sign of homology to any known proteinases was identified; more recent work by Ring PC et al in 2001 has identified a proteolytic activity against N-benzoyl-Phe-Val-Arg-p-nitroanilide associated with this allergen. Fel d1 has now been found to be capable of cleaving gelatin, fibronectin and a synthetic calorimetric trypsin substrate.

The present invention involves applying a proteinase inhibitor to the animal's pelt which thereby inhibits the enzymatic activity of Fel d 1 and other proteolytic allergens, and the allergenic activity/properties.

The proteinase inhibitor used in the present invention should be non-toxic, particularly when ingested by the animal during the cleaning process, and should not itself elicit an allergic reaction. Small inhibitors such as leupeptin (acetyl-Leu-Leu-Arg-CHO) are considered to be most suitable and would be least likely to elicit an allergic response but will still be destroyed in the animal's stomach by pepsin/acid.

The proteinase can be delivered as a dry powder formulation which is applied to the pelt, in the same way as for example flea powder. The inhibitor will become active when taken into solution as the animal licks the pelt during cleaning.

Alternatively, the proteinase inhibitor can be delivered dissolved in a suitable liquid. The inhibitor solution can be applied to the pelt by several methods including: as a bathing solution; or by rubbing it directly into the pelt; or as a spray which is applied to the animal. The above liquid methods of applying the inhibitor can also be combined with a method of removing the allergen if the solution has cleansing activity.

The same or different formulations of the proteinase inhibitor (with or without a cleansing agent) may also be applied to clothing, carpets, curtains, furniture etc to inhibit proteolytic activity therein caused by allergens originally derived from the animal.

### EXAMPLES

Identification of a Proteolytic Activity Extracted from Fur or Present in Saliva.

A clean pet brush was used to brush three common English domestic cats. Fur from guinea pigs, rabbit, dogs and horse was collected in the same way. A different brush was used for each species and between brushings these were cleaned thoroughly in a denaturing detergent solution (Fairy® detergent) and then ethanol. The brushings were transferred to a sterile universal container and extracted by shaking in an extraction buffer, 50 mM Tris.HCl, 5 mM CaCl₂, pH 7.5 (0.1 g/ml). The solution was recovered by centrifugation and stored at −20° C. in aliquots. Saliva was collected from a mastiff-type dog and stored frozen in aliquots until assayed. Sample aliquots were assayed for proteolytic activity using fluorometric blocked peptidylaminomethylcoumarylamide substrates in the cuvette of a Perkin Elmer fluorimeter at 37° C. The fluorimeter was standardised with a known concentration of methylcoumarin, and the assays were designed so that no more than 10% substrate hydrolysis occurred.

Assays were performed in assay buffer; 50 mM Tris.HCl, 5 mM CaCl₂, 0.1% CHAPS, pH 7.5. Data were collected in real-time on a personal computer using the Flusys software package.

In the first series of experiments the sample (12 μl) was assayed against a range of serine and cysteine proteinase aminomethylcoumarylamide (NHMec) substrates (obtained commercially from Bachem) under standard conditions of 5 μM substrate concentration (from a 100-fold stock solution in dimethyl sulfoxide) to give a final volume of 120 μl. After addition of substrate the rate without sample was determined (which in all cases was zero), after which the sample was applied and the progress of the reaction was followed for a further 15 min. The slope obtained (fluorescence vs time) was analysed by linear regression and converted to rate of product formation (nM/min) see results table 2.

The fur samples and saliva sample appear to contain proteolytic activity, more specifically an arginyl endopeptidase activity.

A rough estimate of the Kₘ for the cleavage of Z-Gly-Gly-Arg-NHMec and of Z-Phe-Arg-NHMec by the cat fur enzyme(s) were determined using the same procedure with varying concentrations of these substrates see results table 3.

Kₘ for the cleavage of Z-Gly-Gly-Arg-NHMec was found by non-linear regression analysis using the Enzfitter software package (Elsevier Biosoft): 95±47 μM. Kₘ for the cleavage of Z-Phe-Arg-NHMec was 3.2±0.9 μM. As the activity follows normal Michaelis-Menten kinetics it is clear that it is due to the activity of one or more biological catalysts or enzymes.

Inhibition of the Proteolytic Activity in Animal Fur by Proteinase Inhibitors

The assays were carried out as described above, using 5 μM Z-Gly-Gly-Arg-NHMec as substrate in assay buffer. The assay was begun with substrate and sample and a continuous rate of product formation (nM/min) was measured (v₀) for 15 min. The inhibitor was added to a final concentration of 10 μM in negligible volume from stock solution in dimethyl sulfoxide. The assay was continued for a further 15 min.

Under these conditions reversible inhibition can be measured as the new steady state (v₁) with percentage inhibition being 1−(v₁/v₀)×100. Irreversible inhibition is measured as a second order rate constant and is time dependent but as long as the inhibitor is present in molar excess should always lead eventually to total inhibition. No distinction was made and inhibition is presented as a percentage of full activity. See results table 4.

The results in tables 2-5 are entirely consistent with the proteolytic activity in the fur and saliva samples being due to a trypsin-like serine protease(s).

Effect of Proteinase Inhibitor Treatment on the Allergenicity of the Animal Hair Samples as Judged by Effects on the Response of Cell Lines In Vitro

The identification of the causative agent(s) of an individual's allergic response is conventionally assessed by measuring allergen-specific IgE in patient sera. Such a test is not appropriate for the analysis of IgE-independent allergic events. The possibility of employing a cell line, expressing functional human IgE receptors to test specific cell sensitisation with the serum from an allergic individual or for assessing the direct effect of an allergen presents an attractive alternative to in vivo testing.

Rat basophilic leukaemia, also known as RBL cells were transfected with the IgE-binding domain of the human high affinity receptor complex to establish a permanent mast cell line which permits the contribution made by individual components ie IgE and/or allergens to mast cell responses to be evaluated.

Transfected cells bind human IgE with high affinity and respond to an IgE-mediated antigenic stimulus with the secretion of cellular mediators. Exocytosis can be evaluated by measuring the percent release of either preloaded [3H]5-hydroxytryptamine, or endogenous histamine or β-hexosaminidase. Allergen-triggered cells also induce synthesis and secretion of pro-inflammatory cytokines such as IL4 and IL13. Alternatively the human B-type 8866 cells express CD23, the bioaffinity IgE receptor, which is shed/cleaned following exposure to proteolytic enzymes, including allergenic proteases.

Using these cellular models, it has been established that many allergens including proteases, phospholipases and lectins can induce mediator release in the absence of sensitisation with IgE. The cellular response is in all other respects typical of one involving IgE, however as this step is omitted the immune response is more rapid than if cells were sensitised with IgE.

Assessment of IgE-Dependent and IgE-Independent Allergen-Induced Mast Cell Degranulation.

The methods for the identification of IgE-mediated allergen-induced and non-IgE-mediated allergen induced cell responses employed in the challenge with allergen have been described in the literature.

IgE-mediated, allergen-induced mast cell response was assessed by incubating the cell lines for 12-14 h with IgE purified from the serum of an allergic individual before challenge with increasing concentrations of cognate allergens. Mediator release under these conditions represents cell activation due to an IgE receptor-mediated allergic stimulus and cellular activation by allergen. Cells sensitised with allergen-specific IgE also respond with mediator release when challenged with enzymatically inactive or partially degraded forms of the allergens, where epitopes recognised by IgE have remained intact.

Non-IgE mediated allergen induced response was assessed by measuring IL4 or CD23 released into the culture medium by unsensitised transfected RBL cells exposed to fur allergen or House dust mite allergen or papain (positive controls), with and without prior treatment of the allergen with a proteinase inhibitor.

Cell culture: Adherent cells grown in culture for 3 days in 9 cm plates, were incubated with 5 ml cell dissociation solution (5 min 37° C.), and counted using a haemocytometer.

The cells were pelleted by centrifugation at 1000 rpm for 3 mins and resuspended in medium (DMEM, P/S, FCS, Genetcin) at a density of 0.5×10⁶ cells/ml.

Both unsensitised and sensitised cells (1:500 Serotec IgE) were plated onto 96 well plates (100 μl of cells/well) and left to attach and grow overnight at 37° C., 5% CO₂.

Release of IL4: Cells were checked for confluency the next day, and washed with 200 μl of incubation buffer (without 0.1% BSA) twice, before equilibration to 37° C. in 100 μl of incubation buffer for 10 minutes.

Fresh allergen samples were prepared daily and kept on ice until needed when they were warmed to ambient temperature immediately before use.

Allergen Samples tested:

| | |
|---|---|
| Animal hair extracts | 1:10 and 1:100 dilutions in incubation |
| | buffer - BSA |
| House dust mite | 1:5, 1:10, 1:100 dilutions in incubation |
| (preparation 5-7-1) | buffer - BSA |
| Papain | 2 × crystallised from Sigma activated no |
| (50 μ1 TX100/10 ml SB) | release RBL |
| for total cell lysis | |
| Siraganian buffer | Background |
| Anti IgE | 1:500 (positive for sensitised release) |

100 μl of sample was incubated on the cells in triplicate for a defined time period (15 minutes, 30 minutes or 1 hour).

After the time period the supernatant was carefully removed and clarified by centrifugation (100 rpm 5 mins) to remove any cells that had dissociated before storage on ice.

IL4 was assayed using a commercial ELISA (RDI a diaclone RAT a IL4 Kit and other suppliers), and soluble CD23 using either MUM6 Western blot (tail) or B46 (head).

Assessment of Allergen Induced Inflammation In Vivo: Skin Prick Test of Mice.

The skin prick test is a suitable assay for the allergenic activity of a sample because it measures the systemic type 1 response to challenge.

Mice, 6 to 8 weeks old, were sensitised by i.p. injections of 100 μg allergen protein per mouse on day 0 and 5. Ten to fourteen days after the last injections the mice were challenged by intra-dermal injection of 1 μg allergen protein (skin prick test) into a shaved area of the skin. Unsensitized mice were also challenged similarly.

Reference: Mouton D. et al., Eur. J. Immunol. 18:41-49, 1988; Zhang et al., Hum. Mol.

Gen. 8:601-605, 1999; Kumagai et al., J. Immunol. 4212-4219, 1999.

The diameter of the inflamed area was measured 24 hours after the intra-dermal injection and the affected area calculated.

A portion of the samples used for the skin prick test were incubated with protease inhibitors (10 μM final concentration) for 15 minutes on ice prior to injection.

Overview of the experiment:

| Mice | Allergen | Inhibitor |
|---|---|---|
| Sensitised | Extraction buffer | No |
| Sensitised | Fur extract protein | No |
| Sensitised | Fur extract protein | Yes |
| Sensitised | House dust mite | No |
| Sensitised | House dust mite | Yes |
| Sensitised | Papain | No |
| Sensitised | Papain | Yes |
| Sensitised | Dog saliva | No |
| Sensitised | Dog saliva | Yes |
| Un-sensitised | Extraction buffer | No |
| Un-sensitised | Fur extract protein | No |
| Un-sensitised | Fur extract protein | Yes |
| Un-sensitised | House dust mite | No |
| Un-sensitised | House dust mite | Yes |
| Un-sensitised | Papain | No |
| Un-sensitised | Papain | Yes |
| Un-sensitised | Dog saliva | No |
| Un-sensitised | Dog saliva | Yes |

Allergen:

Animal hair extracts from cat, guinea pigs, rabbit, dogs and horse.

House dust mite (preparation 5-7-1)

Papain

Dog saliva from a mastiff-type dog

Extraction buffer (50 mM Tris.HCl, 5 mM CaCl₂, pH 7.5)

Protease Inhibitors:

ACITIC

Antipain

Aprotinin

Leupeptin

PhCH₂NHCONH-CiTPrOIC

H-Glu-Gly-Arg-chloromethane

The protease inhibitors reduced the inflammatory response to the allergens as compared to the response to allergen protein alone.

Contacting allergens in animal hair and saliva or allergens from house dust mites with protease inhibitors will reduce the allergic response.

Separation of the Arginyl Endopeptidase Activity in Cat Fur from Antigenic Fel d1

We propose that the protein sequenced and described by Morgenstern et al. as Fel d1 is in fact not the major allergen in cat dander, but rather that the allergenic serine proteinase was co-purified with Fel d1. In order to demonstrate that the proteolytic activity is distinct from Fel d1 an extract from cat fur was passed down a column of aprotinin coupled to Sepharose beads. This process removed the proteolytic activity from the sample but did not remove Fel d1, assayed by ELISA. The material bound to the column was released by modulating the pH of the column buffer. This material was shown to contain arginyl endopeptidase activity but to be free from antigenic Fel d1.

Moreover isolate Fel d1 was isolated from the samples by immunoaffinity chromatography using anti-Fel d1 Ig. The process removed Fel d1 but not the proteolytic and allergenic activity from the samples.

Scope of the Inventive Concept

Embraced within the overall inventive concept of the use of hydrolytic enzyme inhibitors for the inactivation of allergens are a number of linked inventions:

The inhibitors may be used for the manufacture of medicaments for the prophylactic treatment of allergic conditions.

The inhibitors may be bound to supporting media (including air filters) to be brought into contact with air during use of such an air treatment device for inactivating airborne allergens. Methods of binding the inhibitors will be readily apparent to the skilled addressee by the application existing knowledge and routine experimentation. Air treatment devices according to this invention would find application in situations including vacuum cleaners (to inactivate allergens found in carpets) and air exhaust filters from tumble dryers (to inactivate allergenic residues released from fabrics). For some inhibitors, it will be preferable to activate the inhibitory activity by maintaining the supported inhibitors in a moist environment. In many applications, such as their use on the exhaust of tumble dryers, this may be provided during the normal course of operation.

Formulations containing the inhibitors may be produced to inactivate allergenic residues on fabrics or carpets. Such formulations, which could conveniently be produced in liquid or powder form, and could optionally include cleaning or fabric conditioning agents, and would find application in situations including: post-wash treatments for inactivating allergenic residues on fabrics from washing formulations or from animal sources; treatments for carpets for inactivating allergenic residues from animals; and treatments for fabrics such as clothes, curtains and upholstery for inactivating allergens from animals or from washing formulations.

The linked inventions comprising the overall inventive concept are described in the claims that follow.

TABLE 1
Description of samples

| Species | Sex | Pedigree | Age | Sample | Date | Source |
|---|---|---|---|---|---|---|
| Cat 1 | Female | Tortoise shell | 7 yr. | 1 | 28.6.01 | Fur/Back |
| | | | | 2 | 9.7.01 | |
| | | | | 3 | 10.7.01 | |
| | | | | 4 | 11.9.01 | |
| Cat 2 | Female | black & white | 17 yr | 1 | 28.6.01 | ″ |
| | | | | 2 | 9.7.01 | |
| | | | | 3 | 11.9.01 | |
| Rabbit | Male | Dwarf English | 4 yr | 1 | 13.9.01 | ″ |
| 1 | | | | | | |
| Dog 1 | Male | English Mastiff | 2 yr | 1 | 13.9.01 | |
| | | | | 2 | 13.9.01 | Saliva |
| Cat 3 | Male | Long hair | | 1 | 14.9.01 | Fur/Back |
| Guinea | Female | Chestnut/White | | 1 | 18.9.01 | ″ |
| P 1 | | | | | | |
| G.P. 2 | Female | ″ | | 1 | 18.9.01 | ″ |
| Dog 2 | Male | B1. Labrador | | 1 | 18.9.01 | ″ |
| Horse 1 | Female | | | 1 | 18.9.01 | Mane |
| | | | | 2 | 18.9.01 | Back |

TABLE 2

| | | | Linear |
|---|---|---|---|
| | | Rate | regression |
| | Sample | (nM/min) | coefficient |
| Trypsin substrates | | | |
| Z-Gly-Gly-Arg-NHMec | Cat1 S1 | 0.87 | 0.9983 |
| Z-Gly-Gly-Arg-NHMec | Cat2 S1 | 1.35 | 0.9995 |
| Pyr-Arg-Thr-Lys-Arg-NHMec | Cat1 S1 | 0.58 | 0.9972 |
| Pyr-Arg-Thr-Lys-Arg-NHMec | Cat2 S1 | 0.33 | 0.9905 |
| Suc-Ala-Phe-Lys-NHMec | Cat1 S1 | No activity | |
| Suc-Ala-Phe-Lys-NHMec | Cat2 S1 | No activity | |
| Z-Phe-Arg-NHMec | Cat1 S3 | 43.5 | 0.9992 |
| Z-Phe-Arg-NHMec | Rabbit1 S1 | 3.93 | 0.9994 |
| Z-Phe-Arg-NHMec | Dog1 S1 | 40.6 | 1.0000 |
| Z-Phe-Arg-NHMec | Dog1 S2 | 1786.9 | 1.0000 |
| Z-Phe-Arg-NHMec | Cat3 S1 | 24.5 | 0.9998 |
| Z-Phe-Arg-NHMec | G. pig1 S1 | 9.11 | 0.9999 |
| Z-Phe-Arg-NHMec | G. pig2 S1 | 18.66 | 1.0000 |
| Bz-Phe-Val-Arg-NHMec | Cat1 S3 | 32.1 | 0.9996 |
| Chymotrypsin substrates | | | |
| Suc-Ala-Ala-Pro-Phe-NHMec | Cat1 S1 | 0.04 | 0.8543 |
| Suc-Ala-Ala-Pro-Phe-NHMec | Cat2 S1 | 0.05 | 0.8964 |
| Suc-Ala-Ala-Phe-NHMec | Cat1 S1 | No activity | |
| Suc-Ala-Ala-Phe-NHMec | Cat2 S1 | 0.03 | 0.6497 |
| Elastase substrates | | | |
| Suc-Ala-Ala-Ala-NHMec | Cat1 S1 | No activity | |
| Suc-Ala-Ala-Ala-NHMec | Cat2 S1 | No activity | |
| Suc-Ala-Ala-Pro-Val-NHMec | Cat1 S1 | No activity | |
| Suc-Ala-Ala-Pro-Val-NHMec | Cat2 S1 | No activity | |

TABLE 3
Z-Gly-Gly-Arg-NHMec

| [Substrate] (μM) | Rate (nM/min) | LRC |
|---|---|---|
| 5 | 1.47 | 0.999 |
| 10 | 2.42 | 0.999 |
| 20 | 3.41 | 0.999 |
| 40 | 4.82 | 0.999 |
| 40 | 8.84 | 1.000 |
| 60 | 10.01 | 1.000 |
| 80 | 13.27 | 1.000 |
| 100 | 11.40 | 1.000 |
| 125 | 13.49 | 1.000 |

Kₘ = 95 ± 47 μM

Z-Phe-Arg-NHMec

| | | |
|---|---|---|
| 0.5 | 3.74 | 0.9992 |
| 1.0 | 6.99 | 0.9996 |
| 2.0 | 10.2 | 0.9986 |
| 5.0 | 27.3 | 0.9994 |
| 60 | 35.4 | 1.0000 |
| 80 | 32.3 | 1.0000 |
| 100 | 33.7 | 0.9999 |

Kₘ = 3.2 ± 0.9 μM

TABLE 4

| | | Substrate | | | % |
|---|---|---|---|---|---|
| Inhibitor | Sample | [*] | v₀ | v₁ | Inhibition |
| E64 [L-trans epoxysuccinyl- | Cat1 S3 | 1 | 2.518 | 2.005 | 20.4 |
| leucylamido-4-guanidino-butane] - | | | | | |
| inhibitor of cysteine proteinases | | | | | |
| 3,4-Dichloroisocoumarin - serine | Cat1 S3 | 1 | 2.845 | 1.837 | 35.4 |
| proteinases with particular efficacy | | | | | |
| against elastase-like enzymes | | | | | |
| Leupeptin - inhibitor of cysteine | Cat1 S3 | 1 | 2.912 | 0 | 100 |
| proteinases and trypsin-like serine | | | | | |
| proteinases inhibitor | | | | | |
| Aprotinin - trypsin-like serine | Cat1 S3 | 1 | 2.805 | 0.292 | 89.6 |
| proteinases | | | | | |
| ACITIC [7-amino-4-chloro-3-(-3- | Cat1 S3 | 1 | 2.745 | 0.002 | 100 |
| isothiureido-propoxy) isocoumarin] - | | | | | |
| trypsin-like serine proteinases | G.pig2 | 2 | 18.66 | 0.964 | 94.8 |
| | S1 | | | | |
| | Horse1 | 2 | 0.782 | 0 | 100 |
| | S1 | | | | |
| | Horse1 | 2 | 0.988 | 0 | 100 |
| | S2 | | | | |
| | Dog2 S1 | 2 | 0.638 | 0.184 | 71.2 |
| PhCH₂NHCONH-CiTPrOIC - | Cat1 S3 | 2 | 5.785 | 0 | 100 |
| trypsin-like serine proteinases | | | | | |
| H-Glu-Gly-Arg-chloromethane - | Cat1 S3 | 1 | 10.844 | 0.143 | 98.7 |
| trypsin-like serine proteinases and | | | | | |
| cysteine proteinases | | | | | |

*Substrate 1; Z-Gly-Gly-Arg-NHMec: Substrate 2; Z-Phe-Arg-NHMec

TABLE 5
Kₗ for the inhibition of Cat1 S3 by leupeptin

| [I] (nM) | V₀ | V₁ | % Inhibition |
|---|---|---|---|
| 0.5 | 8.0 | 6.0 | 25.0 |
| 1.0 | 7.4 | 5.6 | 24.3 |
| 5.0 | 7.9 | 4.9 | 37.9 |
| 10.0 | 9.4 | 4.4 | 53.2 |
| 50.0 | 8.3 | 2.6 | 68.7 |
| 100.0 | 8.2 | 1.8 | 78.0 |

Kₗ was found using the Enzfitter software package: 2.64 ± 1.18 nM

1-18. (Canceled).

19. An air treatment device for inactivating airborne allergens comprising a hydrolytic enzyme inhibitor bound to a supporting medium, such that said supporting medium may be brought into contact with the air during use.

20. An air treatment according to claim 19 wherein the hydrolytic enzyme inhibitor is a protease inhibitor.

21. An air treatment device according to claim 19 wherein the hydrolytic enzyme inhibitor inhibits arginyl endopeptidase activity.

22. An air treatment device according to claim 19 wherein the hydrolytic enzyme inhibitor inhibits serine proteinase activity.

23. A method of using an air treatment device according to claim 19 wherein the said supporting medium is maintained in a moist environment during use.

24. The use of a hydrolytic enzyme inhibitor for the manufacture of a medicament for the prophylactic treatment of allergic conditions.

25. The use as claimed in claim 24 wherein the hydrolytic enzyme inhibitor is a proteinase inhibitor.

26. The use as claimed in claim 24 wherein the allergic reaction is selected from the group comprising: asthma, eczema, hay fever, allergic rhinitis and anaphylaxis.

27. The use as claimed in claim 24 wherein the hydrolytic enzyme inhibitor inhibits arginyl endopeptidase activity.

28. The use as claimed in claim 24 wherein the hydrolytic enzyme inhibitor inhibits serine proteinase activity.

29. A formulation for inactivating allergenic residues on fabrics or carpets comprising a hydrolytic enzyme inhibitor.

30. The formulation of claim 29 wherein the hydrolytic enzyme inhibitor is a proteinase inhibitor.

31. The formulation of claim 29 wherein the hydrolytic enzyme inhibitor inhibits serine proteinase activity.

32. The formulation of claim 29 wherein the hydrolytic enzyme inhibitor inhibits arginyl endopeptidase activity.
